(19) United States
(12) Patent Application Publication
(10) Pub. No.: US 2024/0325540 A1
(43) Pub. Date: Oct. 3, 2024

(54) METHOD OF ENHANCING HUMAN IMMUNITY THROUGH SOLID WATER PARTICLES

(71) Applicant: Shui Yin Lo, Arcadia, CA (US)

(72) Inventor: Shui Yin Lo, Arcadia, CA (US)

(21) Appl. No.: 18/126,672

(22) Filed: Mar. 27, 2023

(51) Int. Cl. A61K 41/00; A61K 9/00; A61K 9/08; A61K 33/00

(52) U.S. Cl. CPC A61K 41/0047; A61K 9/0009; A61K 9/08; A61K 33/00

(57) ABSTRACT

A method for enhancing human immunity through administering a predetermined dosage of a solid water particle solution every day. When water solution that contains solid water particles are absorbed to the blood, the large electric field from the solid water particles will stimulate both the red blood cells the white blood cells to move quickly and excrete cytokines. The method developed by the applicant utilizes that when an aqueous solution containing solid water particles is absorbed into the blood, a large electric field from the solid water particles will stimulate red blood cells and white blood cells to move rapidly and produce cytokines. In this way, the content of cytokines in the human body can be increased, thereby enhancing the immune ability of the human body, and at the same time, the metabolism of the human body can be strengthened by speeding up the blood flow.

### FIELD OF THE INVENTION

The present invention relates generally to a method for solid water particle treatment of the human body. More so, a method for enhancing human immunity through administering a predetermined dosage of a solid water particle solution every day. This will help increasing the blood flow velocity and the amount of the cytokines in the human body.

### BACKGROUND OF THE INVENTION

The following background information may present examples of specific aspects of the prior art (e.g., without limitation, approaches, facts, or common wisdom) that, while expected to be helpful to further educate the reader as to additional aspects of the prior art, is not to be construed as limiting the present invention, or any embodiments thereof, to anything stated or implied therein or inferred thereupon.

In general, the increase of human immune function is measured by the increase of some cytokines. Cytokines are small proteins that are crucial in controlling the growth and activity of other immune system cells and blood cells. Cytokines function as chemical messengers in the immune system. The immune system is a network with several parts that work together to protect your body from threats, like bacteria and virus that can make you sick. It contains immune cells that fight invading pathogens (like viruses and bacteria), allergens and other harmful substances that enter your body. Cytokines signal those immune cells to tight the invaders. Cytokines are important in health and disease, specifically in host immune responses to infection, inflammation, trauma, sepsis, cancer, and reproduction.

However, there is currently no way to effectively use cytokines to boost the human immune system. Therefore, after years of research, the applicant has found a way to strengthen human immunity by stimulating the rapid movement of red blood cells and white blood cells and producing cytokines.

### SUMMARY

In order to solve the problem above, the present invention redefines the way of utilizing cytokines to enhance human immunity.

It is, therefore, an object of the present invention to provide a method that can utilize an electric field to stimulate red blood cells and white blood cells to move rapidly and produce cytokines, while this method is completely harmless to the human body.

It is another object of the present invention to provide a new way of utilizing solid water particle treatment to increase the self-repair ability of the human body.

To achieve the objects above, the present invention provides a method of enhancing human immunity through solid water particles, the method comprising:
(a) detect blood flow velocity and content of cytokines in blood of a human body;
(b) determining whether the blood flow velocity is lower than a first preset value or whether the content of cytokines in blood is lower than a second preset value;
(c) administering a predetermined dosage of a solid water particle solution every day if the blood flow velocity is lower than the first preset value or the content of cytokines in blood is lower than the second preset value, and continue for 14 days to complete a course of treatment;
(d) after the course of treatment is over, the blood flow velocity and the content of cytokines in blood will be detected in the human body;
(e) determining if the blood flow velocity is higher than the first preset value and whether the content of cytokines in blood is higher than the second preset value.

In another aspect, wherein the predetermined dosage of a solid water particle solution is at least 60 ml.

In another aspect, wherein the predetermined dosage of a solid water particle solution is 60 ml to 120 ml.

In another aspect, wherein when the content of cytokines in blood is lower than ⅔ of the second preset value, the predetermined dosage of a solid water particle solution is 120 ml to 180 ml.

In another aspect, wherein when the content of cytokines in blood is lower than ½ of the second preset value, the predetermined dosage of a solid water particle solution is 240 ml to 300 ml.

In another aspect, wherein size of every solid water particle contained in the solid water particle solution is 30 nm˜1 microns.

In another aspect, wherein when the blood flow velocity or the content of cytokine in blood measured in step (e) is still lower than the first preset value or the second preset value, repeat steps (c) to (e) until the blood flow velocity is higher than the first preset value and the content of cytokine in blood is higher than the second preset value.

In another aspect, wherein when the blood flow velocity is detected, the blood vessels at the finger ends are detected, and the first preset value is about 0.1 mm/s.

In another aspect, wherein the cytokine is IFN-γ.

In another aspect, wherein the cytokine is TNF-α, IL-6, and/or IL-12.

In another aspect, wherein the course of treatment is through absorption of solid water particle solution into the blood, electric field of each solid water particle in the solid water particle solution will stimulate red blood cells and white blood cells to move faster and produce cytokines.

Through the above-mentioned method, the present invention can enhance the immunity of the human body by taking a predetermined dosage of a solid water particle solution and using such a method will not cause any harm to the human body. The method developed by the applicant utilizes that when an aqueous solution containing solid water particles is absorbed into the blood, a large electric field front the solid water particles will stimulate red blood cells and white blood cells to move rapidly and produce cytokines. In this way, the content of cytokines in the human body can be increased, thereby enhancing the immune ability of the human body, and at the same time, the metabolism of the human body can be strengthened by speeding up the blood flow.

Other systems, devices, methods, features, and advantages will be or become apparent to one with skill in the art upon examination of the following drawings and detailed description. It is intended that all such additional systems, methods, features, and advantages be included within this description, be within the scope of the present disclosure, and be protected by the accompanying claims and drawings.

### BRIEF DESCRIPTION OF THE DRAWINGS

The invention will now be described, by way of example, with reference to the accompanying drawings, in which:

FIG. 1 illustrates a flowchart diagram of an exemplary method for solid water particle treatment to enhance human immunity, in accordance with an embodiment of the present invention.

Like reference numerals refer to like parts throughout the various views of the drawings.

### DETAILED DESCRIPTION OF THE INVENTION

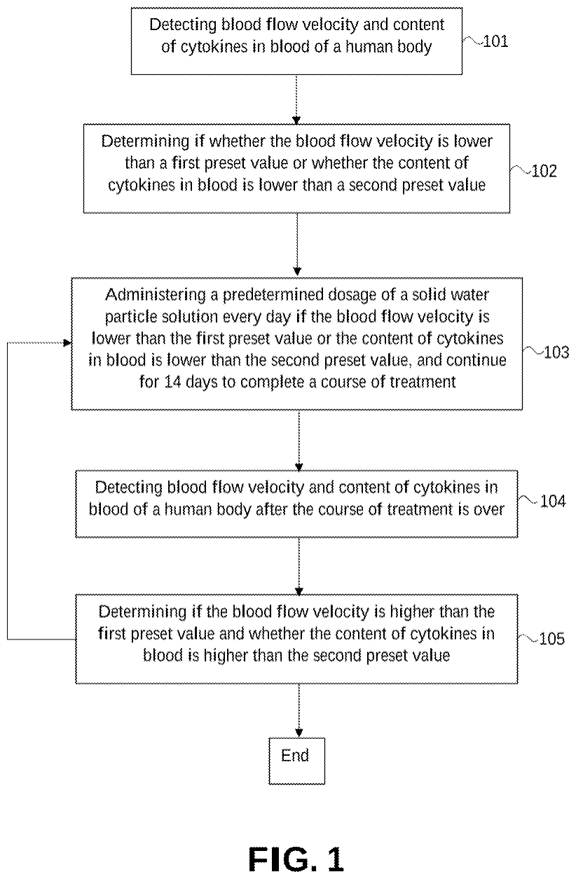

The following detailed description is merely exemplary in nature and is not intended to limit the described embodiments or the application and uses of the described embodiments. As used herein, the word “exemplary” or “illustrative” means “serving as an example, instance, or illustration.” Any implementation described herein as “exemplary” or “illustrative” is not necessarily to be construed as preferred or advantageous over other implementations. All of the implementations described below are exemplary implementations provided to enable persons skilled in the art to make or use the embodiments of the disclosure and are not intended to limit the scope of the disclosure, which is defined by the claims. For purposes of description herein, the terms “upper,” “lower,” “left,” “rear,” “right,” “front,” “vertical,” “horizontal,” and derivatives thereof shall relate to the invention as oriented in FIG. 1. Furthermore, there is no intention to be bound by any expressed or implied theory presented in the preceding technical field, background, brief summary or the following detailed description. It is also to be understood that the specific devices and processes illustrated in the attached drawings, and described in the following specification, are simply exemplary embodiments of the inventive concepts defined in the appended claims. Specific dimensions and other physical characteristics relating to the embodiments disclosed herein are therefore not to be considered as limiting, unless the claims expressly state otherwise.

A method 100 for enhancing human immunity through solid water particles is referenced in FIG. 1. The method 100 for enhancing human immunity through solid water particles, hereafter “method 100” is based on increasing the content of the cytokines and the blood flow velocity through administering the solid water particles.

FIG. 1 references an exemplary flowchart of the method 100 for solid water particle treatment to enhance human immunity, in accordance with an embodiment of the present invention. The method 100 comprising the following steps.

Step (a) 101: detecting blood flow velocity and content of cytokines in blood of a human body. In this step, we must first know the blood flow velocity and cytokines content of the subject before we can decide whether to proceed with the following steps. In this example, we use blood drawing for obtaining the cytokines content of the subject, and the blood flow velocity is measured by the blood vessels at the finger ends, but there are also other measurement methods, so it is not limited to this embodiment.

Step (b) 102: determining whether the blood flow velocity is lower than a first preset value or whether the content of cytokines in blood is lower than a second preset value. After measuring the blood flow velocity and cytokines content, these data will be compared with the preset values of these two items (that is, the human body should have the average value of blood flow velocity and cytokines content). Through comparison, we can clearly know whether the immunity of the tested person is relatively insufficient. In this example, the first preset value is about 0.1 mm/s, which is the average flow velocity of blood in the blood vessels at the finger ends.

Step (c) 103: administering a predetermined dosage of a solid water particle solution every day if the blood flow velocity is lower than the first preset value or the content of cytokines in blood is lower than the second preset value, and continue for 14 days to complete a course of treatment. If it is determined in the previous step that the subject may have insufficient immunity, he/she will be asked to take the predetermined dosage of a solid water particle solution every day for 14 days. When the solid water particle is absorbed by the blood, it stimulates both the red blood cells and the white blood cells to move quickly and produce cytokines through the solid water particles' own large electric field. Through such a basic course of treatment, solid water particle solution can stimulate the human body's self-healing ability, thereby improving their own immunity and making them healthier. In this embodiment, the predetermined dosage of a solid water particle solution is at least 60 ml, and the preferred range is 60 ml to 120 ml, but it can also be other values and is not limited thereto. Moreover, the size of every solid water particle contained in the solid water particle solution can be 30 nm˜1 micron. The electric field generated by such a solid water particle can best stimulate the red blood cells the white blood cells, and at the same time, it will not affect other cells too much.

In addition, if the content of cytokines in the blood is lower than ⅔ of the second preset value, the subject being tested will be deemed as having insufficient immunity, so the predetermined dosage of a solid water particle solution that needs to be taken every day will be increased to 120 ml to 180 ml to ensure that the 14-day course of treatment can still produce sufficient results. If the content of cytokines in the blood is lower than ½ of the second preset value, the subject will be judged as having very weak immunity, so the predetermined dosage of a solid water particle solution that needs to be taken every day will continue to increase to 240 ml to 300 ml to ensure that the 14-day course of treatment can still produce sufficient results.

Step (d) 104: detecting blood flow velocity and content of cytokines in blood of a human body after the course of treatment is over. After the course of treatment is over, the blood flow velocity and cytokines content of the subjects are measured again to determine whether the course of treatment is effective.

Step (e) 105: determining if the blood flow velocity is higher than the first preset value and whether the content of cytokines in blood is higher than the second preset value. After re-measurement of blood flow velocity and cytokines content, compare again as in step (b) to confirm whether the immunity of the subject is similar to other people (close to the average value).

If the blood flow velocity or the content of cytokine in blood measured in step (e) 105 is still lower than the first preset value or the second preset value, repeat steps (c) 103 to (e) 105 until the blood flow velocity is higher than the first preset value and the content of cytokine in blood is higher than the second preset value. Such steps can ensure that the immunity of the subject will not be too low.

Through the above-mentioned method, the immune system of the human body can indeed be enhanced by such a method. In addition, in this embodiment, the cytokine is IFN-γ, but the cytokine can also be TNF-α, IL-6, and/or IL-12, and the second preset value(s) will be adjusted corresponding to the type(s) of cytokine chosen to be measured, so it is not limited to this embodiment.

Relevant experimental data is provided below to prove that the method provided by the present invention really has the effect of reaching its claims.

Form 1

| | Collection | Collection | | TNF-A | Interleukin- | Interleukin- | Interferon- |
|---|---|---|---|---|---|---|---|
| Name | date | time | Pre/Post | (pg/ml) | 1B (pg/ml) | 6 (pg/ml) | γ (pg/ml) |
| JD | Oct. 15, 2007 | 0430 pm | Pre | 2.2 | 2.9 | 9.5 | 20.8 |
| JD | Nov. 30, 2007 | 1155 am | Post | 6.6 | 3.5 | 9.2 | 21.3 |
| DW | Oct. 16, 2007 | 1100 am | Pre | <1.0 | <1.0 | 4.3 | 19.5 |
| DW | Nov. 29, 2007 | 0935 pm | Post | <1.0 | <1.0 | <1.0 | 21.5 |
| TS | Oct. 16, 2007 | 1145 am | Pre | <1.0 | <1.0 | 1.9 | 21 |
| TS | Nov. 30, 2007 | 1255 pm | Post | 1.5 | <1.0 | 3.6 | 22.5 |
| PT | Oct. 16, 2007 | 0500 pm | Pre | <1.0 | <1.0 | 4.5 | 19.8 |
| PT | Nov. 30, 2007 | 0620 pm | Post | <1.0 | <1.0 | 3.9 | 20.3 |
| ER | Oct. 19, 2007 | 1150 am | Pre | <1.0 | <1.0 | 4.1 | 20.5 |
| ER | Nov. 29, 2007 | 1120 am | Post | <1.0 | <1.0 | 5.5 | 21.3 |
| LL | Oct. 19, 2007 | 0140 pm | Pre | <1.0 | 4.6 | <1.0 | 20.5 |
| LL | Nov. 30, 2007 | 0935 am | Post | 2 | 1.6 | <1.0 | 22.3 |
| JP | Oct. 19, 2007 | 0200 pm | Pre | <1.0 | 2.1 | 3.9 | 22 |
| JP | Nov. 30, 2007 | 1130 am | Post | 1.8 | 7.1 | 3.4 | 22 |
| NS | Oct. 19, 2007 | 0210 pm | Pre | <1.0 | 6.1 | 2.4 | 19.3 |
| NS | Nov. 30, 2007 | 1135 am | Post | <1.0 | <1.0 | 1.8 | 21.8 |
| CS | Oct. 20, 2007 | 0630 pm | Pre | <1.0 | <1.0 | 5.3 | 21.3 |
| CS | Nov. 30, 2007 | 0620 pm | Post | 1.4 | 2.9 | 3.8 | 21.8 |

Through the experimental data in Form 1, it can be seen that taking solid water particle solution according to the method of the present invention will greatly increase TNF-A and IFN-γ (both belong to cytokine). Therefore, the present invention can indeed produce a significant enhancement of immune function in human beings. The implications for immune health are obvious.

In some embodiments, the solid water particle solution can be taken orally or by intravenous injection, so that the human body can more easily absorb the solid water particles therein. The oral administration of the solid water particles comprises a liquid, a pill, or a gel consisting of the predetermined quantity of solid water particles.

In one embodiment, the solid water particles consist of only pure water molecules and no other chemicals, and generally does not have harmful side effects. The solid water particle may include, without limitation, stable water clusters, and I_{E}. Those skilled in the art will recognize that solid water particles may be referred to in different scientific names, such as stable water clusters and I_{E} crystals.

These and other advantages of the invention will be further understood and appreciated by those skilled in the art by reference to the following written specification, claims and appended drawings.

Because many modifications, variations, and changes in detail can be made to the described preferred embodiments of the invention, it is intended that all matters in the foregoing description and shown in the accompanying drawings be interpreted as illustrative and not in a limiting sense. Thus, the scope of the invention should be determined by the appended claims and their legal equivalence.

What is claimed is:

1. A method of enhancing human immunity through solid water particles, the method comprising:
(a) detecting blood flow velocity and content of cytokines in blood of a human body;
(b) determining whether the blood flow velocity is lower than a first preset value or whether the content of cytokines in blood is lower than a second preset value;
(c) administering a predetermined dosage of a solid water particle solution every day if the blood flow velocity is lower than the first preset value or the content of cytokines in blood is lower than the second preset value, and continue for 14 days to complete a course of treatment;
(d) detecting blood flow velocity and content of cytokines in blood of a human body after the course of treatment is over;
(e) determining if the blood flow velocity is higher than the first preset value and whether the content of cytokines in blood is higher than the second preset value.

2. The method of claim 1, wherein the predetermined dosage of a solid water particle solution is at least 60 ml.

3. The method of claim 1, wherein the predetermined dosage of a solid water particle solution is 60 ml to 120 ml.

4. The method of claim 1, wherein when the content of cytokines in blood is lower than ⅔ of the second preset value, the predetermined dosage of a solid water particle solution is 120 ml to 180 ml.

5. The method of claim 1, wherein when the content of cytokines in blood is lower than ½ of the second preset value, the predetermined dosage of a solid water particle solution is 240 ml to 300 ml.

6. The method of claim 1, wherein size of every solid water particle contained in the solid water particle solution is 30 nm˜1 micron.

7. The method of claim 1, wherein when the blood flow velocity or the content of cytokine in blood measured in step (e) is still lower than the first preset value or the second preset value, repeat steps (c) to (e) until the blood flow velocity is higher than the first preset value and the content of cytokine in blood is higher than the second preset value.

8. The method of claim 1, wherein when the blood flow velocity is detected, the blood vessels at the finger ends are detected.

9. The method of claim 8, wherein the first preset value is about 0.1 mm/s.

10. The method of claim 1, wherein the cytokine is IFN-γ.

11. The method of claim 1, wherein the cytokine is TNF-α, IL-6, and/or IL-12.

12. The method of claim 1, wherein the course of treatment is through absorption of solid water particle solution into the blood, electric field of each solid water particle in the solid water particle solution will stimulate red blood cells and white blood cells to move faster and produce cytokines.
